(19) United States
(12) Patent Application Publication
(10) Pub. No.: US 2013/0218233 A1
(43) Pub. Date: Aug. 22, 2013

(54) APPARATUS AND A METHOD FOR PERFORMING A SAFE STIMULATION OF A PERSON

(71) Applicants: Udo Warschewske, Berlin (DE); Lars Fiegenbaum, Potsdam (DE)

(72) Inventors: Udo Warschewske, Berlin (DE); Lars Fiegenbaum, Potsdam (DE)

(21) Appl. No.: 13/807,523

(22) PCT Filed: Jun. 30, 2010
(86) PCT No.: PCT/EP2010/059296

§ 371 Date: Jan. 17, 2013

(51) Int. Cl. A61N 1/372

(52) U.S. Cl. CPC A61N 1/372
USPC 607/45

(57) ABSTRACT

The present invention relates to an apparatus and a method for stimulating a person, wherein said apparatus comprises: at least one stimulation signal generator which generates a stimulation signal which is applied to said person; at least one detector for detecting a physiological signal derived from said person in response to said stimulation signal; and a monitoring unit which controls a safety switch to terminate automatically the application of said stimulation signal to said person, if a critical state of said person is recognized in response to the detected physiological signal.

The invention relates to an apparatus and a method for stimulating a person having a safety mechanism to avoid critical states of a person, in particular epileptic seizures.

The human brain contains billions of nerve cells which communicate with one another by means of axons and dendrites, which carry trains of signal pulses called action potentials. Although the brain is protected by the skull and surrounded by a cerebrospinal fluid and further isolated from the blood stream by a blood brain barrier, the delicate nature of the brain makes it vulnerable to numerous diseases and different types of brain damages in which many cells can be lost. Such cell loss may also come about by other neurodegenerative disorders. Brain disorders can lead to cognitive impairments and other functional changes. Further possible disorders caused by drain damages or brain diseases are movement disorders, memory disorders or other functional neurological disorders. Furthermore, any of the sensory systems such as vision or hearing can be affected by such brain diseases or—in case of vision—retinal diseases.

To treat the humans brain, especially in persons with brain diseases, therapeutic devices have been proposed which perform a stimulation of the brain by means of a stimulation signal. For example, a brain of a person is stimulated with an electrical stimulation signal to cause phosphenes, i. e. a visual sensation similar to lightning flashes. However, with conventional devices there is a danger that brain stimulation of normal subjects or a patient might lead to an epileptic seizure caused by the stimulation procedure, in particular when the brain is already damaged. In fact, flashing lights and also hyperventilation may trigger epileptic seizures. Any epileptic seizure of a person might lead to serious injuries. Furthermore, stress during the treatment of the person can lead to a critical state of the person.

Thus, there may be certain risks when delivering electrical stimulation to the brain and therefore it would be desirable to find a method and means to monitor such risks automatically.

Accordingly, it is an object of the present invention to provide an apparatus and a method for stimulating a person which is able to reduce or avoid the above-mentioned risks for the normal subjects or the patients which may be caused or triggered by such stimulation.

This object is achieved by an apparatus having the features of claim 1.

The invention provides an apparatus for stimulating a person, wherein said apparatus comprises:
at least one stimulation signal generator which generates a stimulation signal which is applied to said person;
at least one detector for detecting a physiological signal derived from said person in response to said stimulation signal; and
   a monitoring unit which controls a safety switch to terminate automatically the application of said stimulation signal to said person, if a critical state of said person is recognized in response to the detected physiological signal.

In an embodiment of the apparatus according to the present invention the stimulation signal generator generates an electrical stimulation signal which is applied via the safety switch to one or several electrodes attachable to a skin, preferable on the head of the person.

In an alternative embodiment of the apparatus according to the present invention the stimulation signal generator generates a visual stimulation signal which is applied to the person by means of a video monitor or an electro-visual eye stimulator.

In a further embodiment the stimulation signal generator generates both an electrical stimulation signal and a visual stimulation signal.

In a further embodiment of the apparatus according to the present invention the monitoring unit processes the detected physiological signal of the person in real time and actuates the safety switch in real time if a critical state of the person is recognized.

In an embodiment of the apparatus according to the present invention the monitoring unit evaluates the detected physiological signals of the person in the time and/or frequency domain.

In a further embodiment of the apparatus according to the present invention the monitoring unit measures a power density of the detected physiological signal for a sliding time window within a selectable frequency band.

In an embodiment of the apparatus according to the present invention the safety switch is actuated by the monitoring unit if the measured power density exceeds an adjustable threshold value.

In a possible embodiment of the apparatus according to the present invention the monitoring unit performs a pattern based and/or statistical evaluation of the detected physiological signal of the respective person.

In a possible embodiment of the apparatus according to the present invention the detector comprises a biofeedback signal detector.

In a possible embodiment of the apparatus according to the present invention the biofeedback signal detector is a EEG signal detector.

In a possible embodiment of the apparatus according to the present invention the biofeedback signal detector is adapted to detect a blood pressure, a skin resistance or an electro-cardiac signal of the respective person.

In an embodiment of the apparatus according to the present invention the monitoring unit recognizes a critical state of the person if at least one configurable criterium is fulfilled.

In an embodiment of the apparatus according to the present invention the monitoring unit processes the detected physiological signal within at least one selectable frequency band.

In a possible embodiment of the apparatus according to the present invention the frequency band is a Beta frequency band having a frequency in the range of 20-40 HZ.

The invention further provides a method for performing a safe stimulation of a person, wherein a stimulation signal applied to the person is terminated automatically if a critical state of the person is recognized in response to a predetermined physiological signal of the respective person.

In the following embodiments of the apparatus and method for safe stimulation of a person are described with reference to the enclosed figures.

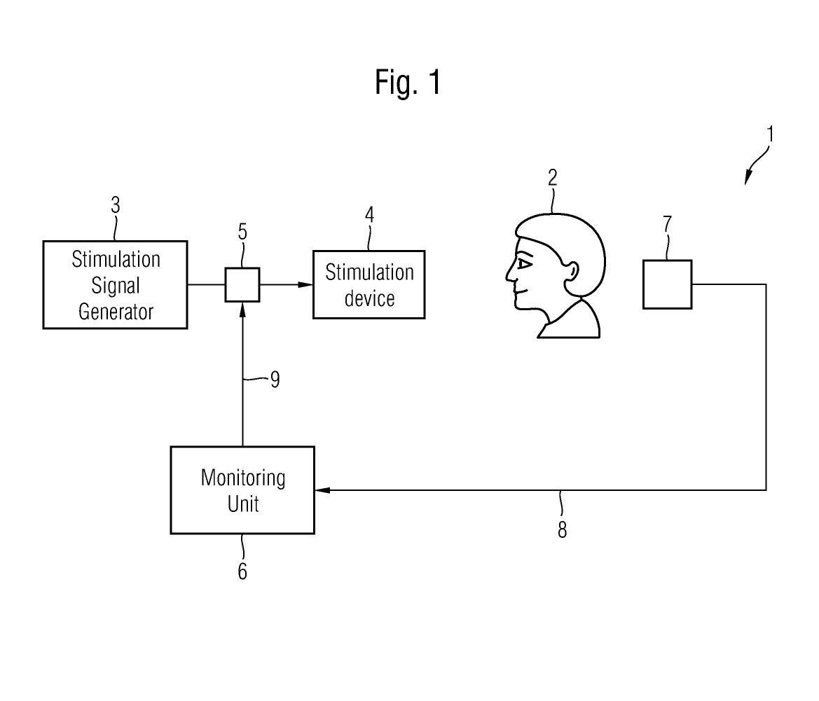

FIG. 1 shows a block diagram for illustrating a possible embodiment of an apparatus for stimulating a person according to the present invention;

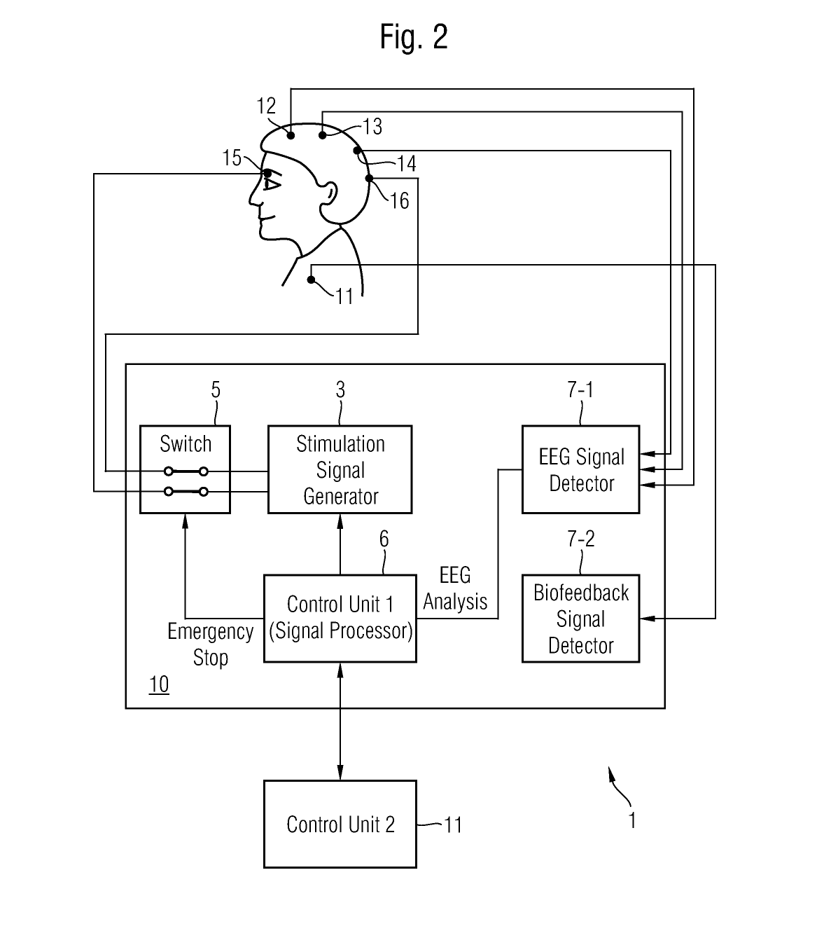

FIG. 2 shows a further block diagram for illustrating a further possible embodiment of an apparatus for stimulating a person according to the present invention;

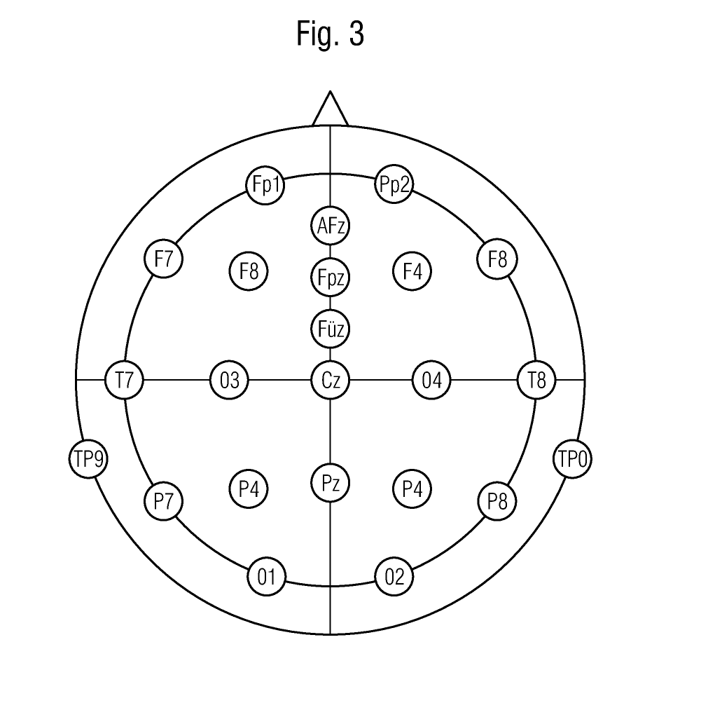

FIG. 3 shows a schematic diagram for illustrating the attachments of electrodes to a skull of a person as employed by the apparatus according to the present invention;

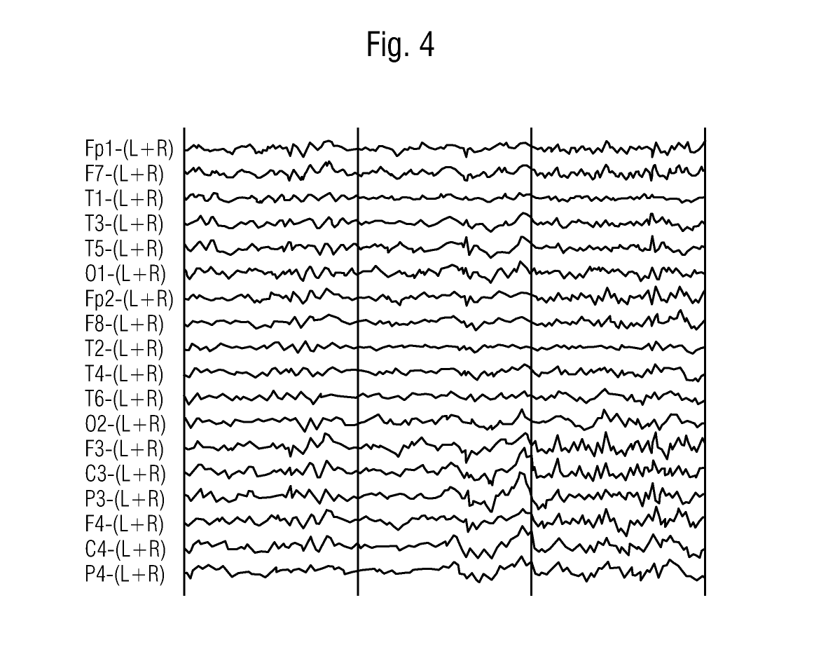

FIG. 4 shows signal diagrams derived from different electrodes attached to a person as employed by the apparatus according to the present invention;

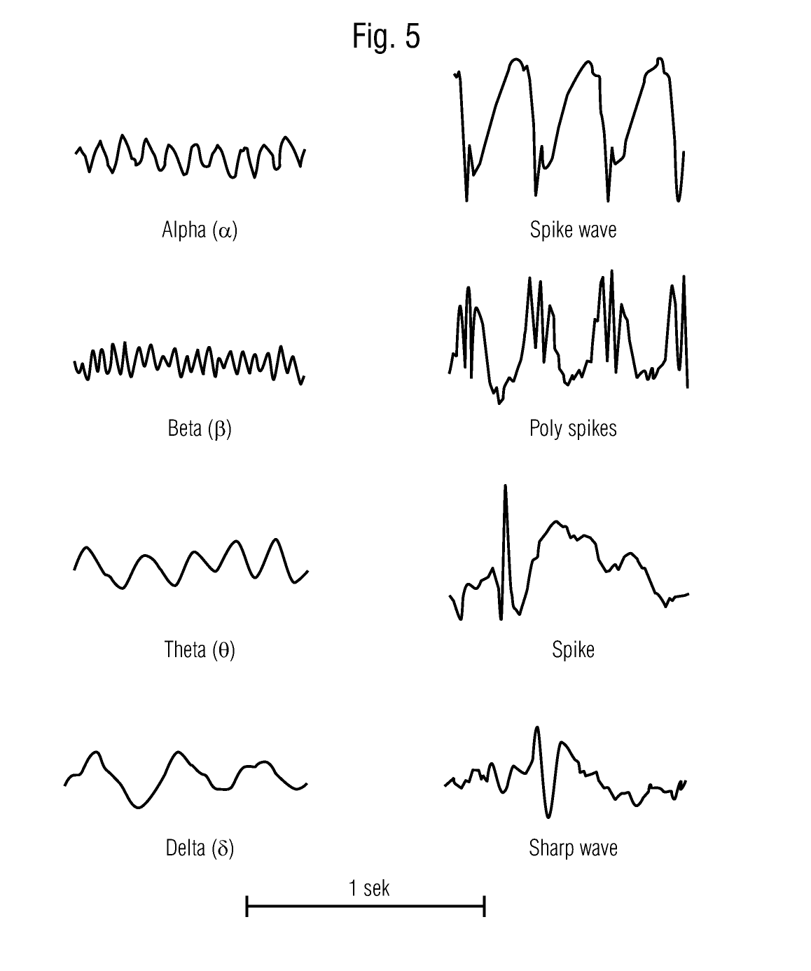

FIG. 5 shows normal potentials and potentials typical for epilepsy in EEG signals derived from a person for different kinds of signal waves;

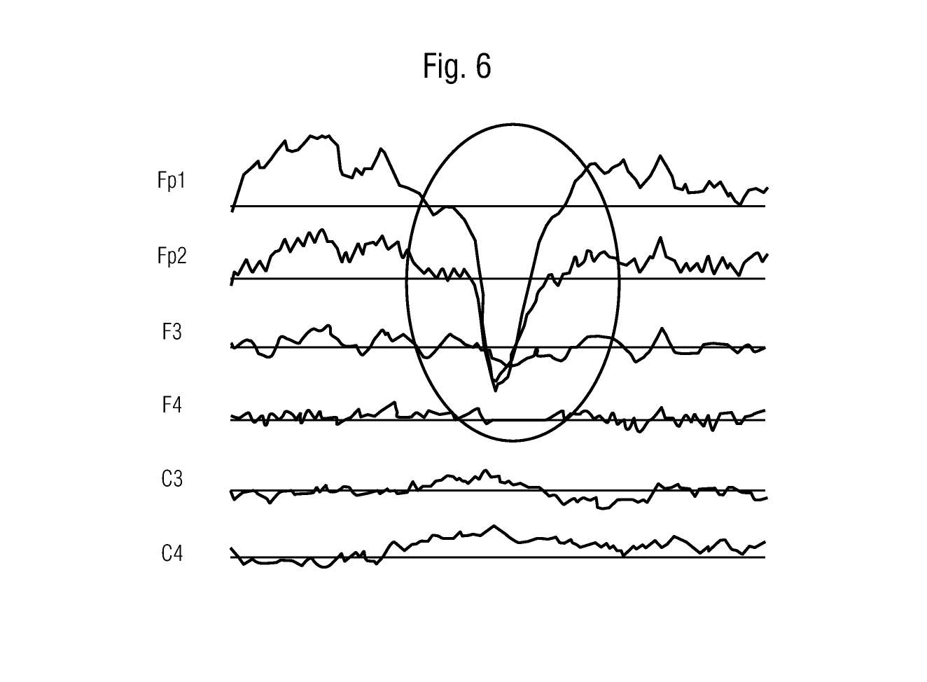

FIG. 6 shows diagrams to illustrate an artefact caused by the movement of the eye of a person during generation of EEG signals.

As can be seen from FIG. 1 an apparatus 1 for stimulating a person 2 comprises at least one stimulation signal generator 3 which generates a stimulation signal. The generated stimulation signal is applied to the person 2. The stimulation signal can be applied by means of a stimulation device 4. This stimulation device 4 can be formed by one or several electrodes attachable to a skin of the person 2. The stimulation device 4 can also be formed by a video monitor or an electro-visual eye stimulator or an audio stimulator providing acoustical stimulation signals. In the shown embodiment of FIG. 2 the stimulation signal generated by the stimulation signal generator 3 is applied to the stimulation device 4 via as safety switch 5. The safety switch 5 is controlled by a monitoring unit 6. Furthermore, the apparatus 1 according to the present invention as shown in FIG. 1 comprises at least one detector 7 for detecting a physiological signal derived from the person 2 in response to the stimulation signal applied to the person 2 by means of the stimulation device 4. The detected physiological signal is applied by the detector 7 via a signal line 8 to the monitoring unit 6. The monitoring unit 6 controls the safety switch 5 via a control signal line 9. The monitoring unit 6 controls the safety switch 5 to terminate automatically the application of the stimulation signal to the person 2, if a critical state of the person 2 is recognized by the monitoring unit 6 in response to the detected physiological signal. In the embodiment shown in FIG. 1 the safety switch 5 can be formed by an electronic element such as a relay. In other embodiments the electronic switch 5 can be formed by a transistor having a control gate connected to the monitoring unit 6. In a further embodiment the safety switch 5 can be integrated in the stimulation signal generator 3. The monitoring unit 6 can terminate the application of the stimulation signal to the person 2 also by switching off the stimulation signal generator 3.

In the embodiments of apparatus 1 according to the present invention the stimulation signal generator 3 generates an electrical stimulation signal which is applied via the safety switch 5 to one or several electrodes 4 attachable to a skin of the person 2. These electrodes can be attached to a head of the person 2 according to a predetermined attachment scheme.

In a further possible embodiment of the apparatus 1 according to the present invention the stimulation signal generator 3 generates a visual or optical stimulation signal which is applied to the person 2 by means of a video monitor or an electro visual eye stimulator forming the stimulation device 4.

The monitoring unit 6 processes the detected physiological signal of the person 2 in real time and actuates the safety switch 5 in real time if a critical state of the person 2 is recognized. In a possible embodiment the monitoring unit 6 evaluates the detected physiological signals of the person 2 in the time domain. In an alternative embodiment of the monitoring unit 6 evaluates the detected physiological signals of the person 2 in the frequency domain. The monitoring unit 6 can for example measure a power density of the detected physiological signal for a sliding time window within a selectable frequency band and then evaluate the physiological signal. In a possible embodiment the safety switch 5 is actuated by the monitoring unit 6 if the measured power density exceeds an adjustable threshold value. This adjustable threshold value can be set by an operator by means of a user interface connected to the monitoring unit 6. In possible embodiments the monitoring unit 6 performs a pattern based evaluation of the detected physiological signal of the person 2. In a further possible embodiment the monitoring unit 6 performs a statistical evaluation of the detected physiological signal of the person 2.

One or several detectors 7 can be provided for detecting a physiological signal of the person 2 in response to the stimulation signal. The detector 7 can comprise a biofeedback signal detector. In a preferred embodiment the biofeedback signal detector is an EEG signal detector. Further biofeedback signal detectors 7 can be provided as well. A bio-feedbacksignal detector 7 can be formed by a detector for measuring a blood pressure of the person 2. A further biofeedback signal detector 7 can be provided for measuring a skin resistance of the person 2. Further, a biofeedback signal detector 7 can be provided for deriving an electro-cardiac signal of the respective person 2. All these biofeedback signal detectors 7 are connected in a possible embodiment via a corresponding signal line to the monitoring unit 6. The monitoring unit 6 can evaluate the detected physiological signals provided by the biofeedback signal detectors 7 to recognize a critical state of the person 2. A critical state of the person 2 can be recognized by the monitoring unit 6 according to at least one configurable criterion. The critical state of the person 2 is recognized if at least one configurable criterion is fulfilled. The monitoring unit 6 recognizes the critical state of the person 2 in a preferred embodiment in advance, i.e. if the person 2 shows symptoms that he will reach a critical state in the near future. For example, the monitoring unit 6 will recognize that the treated person 2 is prone to have an epileptic seizure if further treated with the stimulation signal applied to the person 2 by means of the stimulation device 4.

In a possible embodiment the monitoring unit 6 processes the detected physiological signals such as EEG signals within at least one selectable frequency band FB. The frequency band FB might be selected by an operator by means of a user interface of the apparatus 1. In a possible embodiment the frequency band FB can be a predetermined frequency band for example a Beta frequency band having a frequency in the range of 20-40 HZ. In a possible embodiment the frequency band FB is selected automatically depending on the disorder to be treated or the stimulation signal applied to the person 2. The evaluation of the detected physiological signal can be performed in one or several predetermined frequency bands FB of the EEG signal or by evaluating small cortical potentials SCP.

FIG. 2 shows a block diagram for a possible embodiment of the apparatus 1 for stimulating a person 2 according to the present invention. The embodiment shown in FIG. 2 comprises a person unit 10 forming a therapeutic device which has detectors 7-1, 7-2 for detecting physiological signals from the person 2 in response to a stimulation signal generated by a stimulation signal generator 3 integrated in the person unit 10. The person unit 10 comprises a control unit having at least one signal processor forming the monitoring unit 6. The detectors 7-1, 7-2 are also integrated in the person unit 10 in the shown embodiment. In the embodiment of FIG. 2 the person unit 2 has an integrated safety switch 5 to terminate automatically the application of the stimulation signal to the person 2 when a critical state of the person 2 is recognized by the monitoring unit 6 in response to the detected physiological signals provided by the EEG signal detector 7-1 and a further biofeedback signal detector 7-2. In the embodiment of FIG. 2 the biofeedback signal detector 7-2 receives an electro-cardiac signal of the person 2 from an electrode 11 attached to the body of the person 2. The EEG signal detector 7-1 receives EEG signals from electrodes 12, 13, 14 attached to the head of the person 2. In the embodiment of FIG. 2 an electrical stimulation signal is generated by the signal generator 3 and applied via the safety switch 5 to a stimulation electrode 15 attached close to an eye of the person 2. Furthermore, a reference electrode 16 is also attached to the head of the person 2.

In a further embodiment the person unit 10 comprises an integrated power supply unit. The monitoring unit 6 can comprise an embedded controller such as a signal processor, a solid state memory disk and a RAM. Further, the person unit 10 can comprise a network adapter connecting the person unit 10 to a data network. In a possible embodiment the person unit 10 can also be connected to a person response button which can be actuated by the person 2 in response to stimulation signals. The person unit 10 is connected via the data network to a control unit 11. The control unit 11 can be a remote control unit connected to the person unit 10 via a data network such as the internet. Both the person unit 10 as well as the control unit 11 can comprise a graphical user interface GUI for an operator. The central control unit 11 can record and visualize measurement data of the systems' operational status and can provide tools for data analysis and post processing. The control unit 11 can provide an interface for a user such as a scientist or a physician and can supervise one or more person units 10. The person unit 10 and the central control unit 11 can be connected with each other via a network which can be a wireless or a wired network. The central control unit 11 can comprise a workstation with a mouse and a keyboard or a touchscreen. Several monitors can be provided for displaying a system status and person monitoring data. Furthermore, a non transient storage device for data recording forming a person database can be connected to the central control unit 11.

In a possible embodiment the person unit 10 can be a mobile device and can be attached to the person 2. This mobile device 10 communicates with the central control unit 11 via a wireless network link. For sake of mobility the power supply of the person unit 10 can be a rechargeable battery. Each person unit 10 can comprise a local memory for data processing. This memory can also be used to safe measurement data in case of limitations or dysfunctions in the communication between the person unit 10 and the central unit 11. In a possible embodiment the person unit 10 can comprise a replaceable storage device such as a SD chip.

In the embodiment shown in FIG. 2 the monitoring unit 6 comprises means for recognizing a critical state of the person 2 in response to the detected physiological signals. In an alternative embodiment detection of a critical state of the person 2 can be performed by the central control unit 11. Real time processing can be provided by the central control unit 11. This can include the detection of health critical conditions of the person 2, e. g. a pending epileptic seizure of the person 2. Furthermore, subunits can be controlled directly depending on changing conditions measured by another subunit, e. g. measuring of stimulation pulses in accordance with the EEG wavelet patterns. Furthermore, computing of high level information based on measurement data such as zero crossing or spectrum changes can be performed. Incoming trigger signals can be processed or trigger signals can be generated. To provide flexibility in processing the signal processor the monitoring unit 6 is capable to accept command (scripts) from the central control unit 2. The scripts can be program fragments executable by a processor of the central control unit 11.

The physiological signals derived from the person 2 in response to the stimulation signal are processed in a possible embodiment by signal processor of the monitoring unit 6 within the person unit 10. In an alternative embodiment the detected physiological signals are processed by the central control unit 11. Local processing of the physiological signals such as EEG data by the person unit 10 has the advantage that the safety switch 5 will be actuated by the monitoring unit 6 of the person unit 10 even if the connection between the person unit 10 and the remote control unit 11 fails.

In the embodiment shown in FIG. 2 an electrical stimulation signal is applied to the person 2 by means of the electrode 15. In an alternative embodiment the person 2 is stimulated by an optical stimulation signal which can comprise light pulses generated by LEDs. The light emitting diods LEDs can be arranged in a google close to the person's eyes. Furthermore a person response button can help to receive a person's feedback according to stimulation thresholds or given tasks. The signal generated by the person's response button can be used as a trigger signal to start or stop stimulation processes. Furthermore, the response button signal can be recorded synchroneously with the other measured signals. In a possible embodiment the detected physiological signal can comprise an EEG signal having 32 channels at a high sampling rate.

FIG. 3 shows a possible arrangement for the attachment of stimulation electrodes as well as EEG electrodes to the head of a person 2.

FIG. 4 shows recorded potentials between recording electrodes respectively over time. As can be seen frontal spikes are often found in persons 2 with frontal lobe epilepsy FLE and can appear as fragmented spike and wave discharges in idiopathic generalized epilepsy during drowsiness. There are often spikes with high amplitude broad discharges which can be reflected in the contralateral frontal region. Monitoring the recorded potentials allows that an imminent epileptic state can be predicted by the monitoring unit 6.

FIG. 5 shows normal potentials in EEG signals in contrast to potential spikes for epileptic states in different frequency bands. For example poly spikes in the Beta frequency band can indicate a possible critical state of the respective person 2.

Movement of an eye of a person can cause changes in the measured EEG potentials. However, as shown in FIG. 6 such an eye movement causes mainly changes at the frontal electrodes FP1, FP2, F3 located at the front side of the head of the respective person 2. Accordingly, eye movement can not comprise the detection of a critical state to the monitoring unit 6 of the apparatus 1 according to the present invention.

In general the monitoring unit 6 monitors changes of a state of the brain of the respective person 2. Methods to detect such changes include wavelet or pattern analysis. For example, by means of statistical analysis independent signal components can be detected which allow to search for specific patterns within the signal.

The apparatus 1 according to the present invention increases the safety of the person 2 significantly and avoids the risk of a critical state, in particular epileptic seizures, of the person 2.

1. An apparatus for stimulating a person,
wherein said apparatus comprises:
a) at least one stimulation signal generator which generates a stimulation signal which is applied to said person;
b) at least one detector for detecting at least one physiological signal derived from said person in response to said stimulation signal;
wherein said detector comprises a biofeedback signal detector adapted to detect a physiological signal comprising a blood pressure, a skin resistance or an electrocardiac signal of said person;
c) a monitoring unit which controls a safety switch to terminate automatically the application of said stimulation signal to said person, if a critical state of said person is recognized in response to the detected physiological signal.

2. The apparatus according to claim 1,
wherein said stimulation signal generator generates an electrical stimulation signal which is applied via said safety switch to one or several electrodes attachable to a skin of said person.

3. The apparatus according to claim 1,
wherein said stimulation signal generator generates a visual stimulation signal which is applied to said person by means of a video monitor or an electro-visual eye stimulator.

4. The apparatus according to claim 3,
wherein said monitoring unit processes the detected physiological signal of said person in real time and actuates said safety switch in real time if a critical state of said person is recognized.

5. The apparatus according to claim 4,
wherein said monitoring unit evaluates the detected physiological signals of said person in the time and/or frequency domain.

6. The apparatus according to claim 5,
wherein said monitoring unit measures a power density of the detected physiological signal for a sliding time window within a selectable frequency band.

7. The apparatus according to claim 6,
wherein the safety switch is actuated by said monitoring unit if the measured power density exceeds an adjustable threshold value.

8. The apparatus according to claim 1,
wherein said monitoring unit performs a pattern based and/or statistical evaluation of the detected physiological signal of said person.

9. The apparatus according to claim 1,
wherein said biofeedback signal detector further comprises an EEG signal detector.

10. (canceled)

11. (canceled)

12. The apparatus according to claim 1,
wherein said monitoring unit recognizes a critical state of said person if at least one configurable criterium is fulfilled.

13. The apparatus according to claim 9,
wherein said monitoring unit processes the detected physiological signal within at least one selectable frequency band.

14. The apparatus according to claim 13,
wherein said frequency band is a Beta frequency band having a frequency in the range of 20-40 HZ.

15. A method for performing a safe stimulation of a person,
wherein a stimulation signal applied to said person is terminated automatically if a critical state of said person is recognized in response to a detected physiological signal of said person, wherein the detected physiological signal comprises a blood pressure, a skin resistance or an electro-cardiac signal of said person.
